(19) United States
(12) Patent Application Publication
(10) Pub. No.: US 2021/0290154 A1
(43) Pub. Date: Sep. 23, 2021

(54) METHOD AND SYSTEM FOR DETERMINING AN OPTIMAL SET OF OPERATING PARAMETERS FOR AN AESTHETIC SKIN TREATMENT UNIT

(71) Applicant: LUMENIS LTD., Yokneam (IL)

(72) Inventors: Daniel Gat, Zichron Yaacov (IL); Andrey Gandman, Haifa (IL); Yossi Appelbaum-Elad, Jerusalem (IL); Ben Levy, Yokneam (IL)

(21) Appl. No.: 17/203,994

(22) Filed: Mar. 17, 2021

Related U.S. Application Data
(60) Provisional application No. 62/990,665, filed on Mar. 17, 2020.
(60) Provisional application No. 63/132,554, filed on Dec. 31, 2020.

(51) Int. Cl. A61B 5/00; G06N 3/08; G16H 30/20; G16H 20/40

(52) U.S. Cl. CPC A61B 5/4836; A61B 5/0077; A61B 5/441; A61B 5/7267; A61B 5/0075; A61N 5/0616; A61B 5/0035; G06N 3/08; G16H 30/20; G16H 20/40; A61B 5/7246

(57) ABSTRACT

The present disclosure provides method and system determining an optimal set of operating parameters for desired clinical outcome. The method comprises to receive treatment or target skin data comprising skin characteristics associated with skin to be treated with an aesthetic treatment by the aesthetic skin treatment unit and preset operating parameters for performing the aesthetic treatment. Further, the treatment data is analyzed using plurality of trained models to predict plurality of sets of operating parameters for the aesthetic skin treatment unit to perform the aesthetic treatment. Using the plurality of sets of operating parameters, an optimal set of operating parameters is determined for performing the aesthetic treatment by the using the aesthetic skin treatment unit. By proposed system and method, accurate set of operation parameters may be predicted to achieve desired clinical outcomes, without human intervention.

### RELATED APPLICATIONS

The present application claims the benefit of U.S. provisional application No. 62/990,665 filed 17 Mar. 2020 and U.S. provisional application No. 63/132,554 filed 31 Dec. 2020, assigned to the assignee of the present disclosure, is directed to some features of the Method and System for Determining An Optimal Set of Operating Parameters for an Aesthetic Skin Treatment Unit and is herein incorporated by reference in its entirety.

### TECHNICAL FIELD

The present disclosure generally relates to aesthetic treatment techniques. Particularly, but not exclusively, the present disclosure relates to a method and system for determining optimal parameters for operating an aesthetic skin treatment unit.

### BACKGROUND

Aesthetic treatments and procedures comprise medical procedures that are aimed at improving physical appearance and satisfaction of a patient. An aesthetic treatment focuses on altering aesthetic appearance through the treatment of conditions including scars, skin laxity, wrinkles, moles, liver spots, excess fat, cellulite, unwanted hair, skin discoloration, spider veins and so on. For any aesthetic treatment, generally, several targeted areas on body of the patient are subjected to the aesthetic treatment using energy-based system, such as laser and/or light energy-based systems. In these treatments, light energy with pre-defined parameters may be typically projected on skin tissue area. In general, the treatment procedures may involve manual use of a handpiece or an applicator. The type of energy-based system utilized, and the working or operating parameters of the laser is treatment-dependent as well as physiology-dependent.

In addition, appropriate selection of operating parameters for the energy-based system may be important for satisfactory clinical outcomes. A practitioner may have to consider skin attributes such as skin type, presence of tanning, hair color, hair density, hair thickness, blood vessel diameter and depth, lesion type, pigment depth, pigment intensity, tattoo color and type, to decide laser parameters to be used.

Based on decided operating parameters, the practitioner may need to follow a process of trial-and-error, observing the immediate responses (i.e. “visual end point”) and fine tuning the laser parameters accordingly. The current energy-based systems for therapeutic and aesthetic treatments require a subjective personal estimation of different physiological parameters for choosing the right working parameters of the energy source, followed by manual techniques for laser positioning, aiming and operation.

The information disclosed in this background of the disclosure section is only for enhancement of understanding of the general background of the invention and should not be taken as an acknowledgement or any form of suggestion that this information forms the prior art already known to a person skilled in the art.

### SUMMARY

An objective of the present disclosure relates to a method for a method for determining an optimal set of operating parameters for an aesthetic skin treatment unit, comprising: receiving target skin data comprising at least one skin characteristic associated with skin to be treated with an aesthetic treatment by the aesthetic skin treatment unit: receiving preset operating parameters for performing the aesthetic treatment by the aesthetic skin treatment unit; analyzing the target skin data and the preset operating parameters using a plurality of trained models to predict a plurality of sets of operating parameters for the aesthetic skin treatment unit to perform the aesthetic treatment; and determining an optimal set of operating parameters for performing the aesthetic treatment by the using the aesthetic skin treatment unit, using the plurality of sets of operating parameters.

In another objective the method, wherein the target skin data comprises at least one of pre-treatment skin data, real-time skin data in response to the aesthetic treatment, or any combination thereof. The method, wherein the target skin data is received in a form of at least one of multi-spectral images of the skin, Red Green Blue (RGB) images of the skin, or any combination thereof.

In one objective, the method, wherein the multi-spectral images of the skin are obtained by illuminating light on the skin with a plurality of wavelengths, and by analyzing the multi-spectral images obtained, the one or more trained models are configured to achieve depth analysis of the skin. Also, wherein the plurality of trained models comprises a first model, a second model, a third model and a fourth model, wherein each of the plurality of trained models are pre-trained using index data, pre-defined successful treatment data and pre-defined unsuccessful treatment data, related to the aesthetic treatment.

In yet another objective, the method wherein the first model is a deep-learning classifier model trained using the pre-defined successful treatment data, wherein the second model is a regressor model trained using the index data, the pre-defined successful treatment data, and the pre-defined unsuccessful treatment data, wherein the third model is a gradient boosting model trained using the pre-defined successful treatment data and the index data, and wherein the fourth model is an autoencoder model trained using the index data.

In an objective, the method, wherein analyzing the target skin data using the first model from the plurality of trained models, comprises: classifying the at least one skin characteristic of the target skin data to identify one or more first classes for the at least one skin characteristic; and correlating the one or more first classes with the preset operating parameters, to obtain first set of operating parameters amongst the plurality of sets of operating parameters.

Another objective, the method, wherein analyzing the target skin data using the second model and the third model from the one or more trained models comprises: extracting, using the second model, real-time skin data from the skin target skin data; and correlating, using the third model, the real-time skin data with the preset operating parameters, to obtain second set of operating parameters amongst the plurality of sets of operating parameters. Also, the method, wherein analyzing the target skin data using the first model, the second model and the third model from the one or more trained models comprises: receiving the one or more first classes from the first model; receiving the real time data and one or more second classes obtained by classifying the real-time skin data, from the second model; generating, using the fourth model, encoded representation for the skin data using the index data; generating semantic representation for the target skin data by concatenating the one or more first classes, the real-time skin data, the one or more second classes and the encoded representation; and interpolating information in the semantic representation to obtain a third set of operating parameters from the plurality of sets of operating parameters.

In one objective, the method further comprises one of: providing the optimal set of operating parameters to the aesthetic skin treatment unit, for controlling automated operation of the aesthetic skin treatment unit; displaying the optimal set of the operating parameter to a display unit associated with the aesthetic skin treatment unit, for manually controlling the operation of the aesthetic skin treatment unit. Also, the method wherein providing the optimal set of operating parameters to the aesthetic skin treatment unit comprises: correcting the preset operating parameters for performing the aesthetic treatment by the aesthetic skin treatment unit, in accordance with the optimal set of operating parameters. Further, the method, wherein determining the optimal set of operating parameters comprises: calculating mean value of the plurality of sets of operating parameters to output optimal set of operating parameters.

In one objective of the present disclosure relates to a system for determining an optimal set of operating parameters for an aesthetic skin treatment unit, comprises: a processor; and a memory communicatively coupled to the processor, wherein the memory stores processor-executable instructions, which, on execution, cause the processor to: receive target skin data comprising at least one skin characteristic associated with skin to be treated with an aesthetic treatment by the aesthetic skin treatment unit: receive preset operating parameters for performing the aesthetic treatment by the aesthetic skin treatment unit; analyze the target skin data and the preset operating parameters using a plurality of trained models to predict a plurality of sets of operating parameters for the aesthetic skin treatment unit to perform the aesthetic treatment; and determine an optimal set of operating parameters for performing the aesthetic treatment by the using the aesthetic skin treatment unit, using the plurality of sets of operating parameters.

In another objective, the system, wherein the target skin data comprises at least one of pre-treatment skin data, real-time skin data in response to the aesthetic treatment, or any combination thereof. Also, the system wherein, the target skin data is received in a form of at least one of multi- spectral images of the skin, RGB images of the skin, or any combination thereof. Further, the system wherein, the multi-spectral images of the skin are obtained by illuminating light on the skin with a plurality of wavelengths, and by analyzing the multi-spectral images obtained, the one or more trained models are configured to achieve depth analysis of the skin.

In one objective, the system, wherein the plurality of trained models comprises a first model, a second model, a third model and a fourth model, wherein each of the plurality of trained models are pre-trained using index data, pre-defined successful treatment data and pre-defined unsuccessful treatment data, related to the aesthetic treatment. Also, the system wherein the first model is a deep-learning classifier model trained using the pre-defined successful treatment data, wherein the second model is a regressor model trained using the index data, the pre-defined successful treatment data, and the pre-defined unsuccessful treatment data, wherein the third model is a gradient boosting model trained using the pre-defined successful treatment data and the index data, and wherein the fourth model is an autoencoder model trained using the index data. Further, the system, wherein the processor is configured to analyze the target skin data using the first model from the plurality of trained models by: classifying the at least one skin characteristic of the target skin data to identify one or more first classes for the at least one skin characteristic; and correlating the one or more first classes with the preset operating parameters, to obtain first set of operating parameters amongst the plurality of sets of operating parameters.

In yet another objective, the system, wherein the processor is configured to analyze the target skin data using the second model and the third model from the one or more trained models by: extracting, using the second model, real-time skin data from the skin target skin data; and correlating, using the third model, the real-time skin data with the preset operating parameters, to obtain second set of operating parameters amongst the plurality of sets of operating parameters. Also, the system, wherein the processor is configured to analyze the target skin data using the first model, the second model and the third model from the one or more trained models by: receiving the one or more first classes from the first model; receiving the real-time data and one or more second classes obtained by classifying the real-time skin data, from the second model; generating, using the fourth model, encoded representation for the skin data using the index data; generating semantic representation for the target skin data by concatenating the one or more first classes, the real-time skin data, the one or more second classes and the encoded representation; and interpolating information in the semantic representation to obtain a third set of operating parameters from the plurality of sets of operating parameters.

The system further comprises the processor configured to: provide the optimal set of operating parameters to the aesthetic skin treatment unit, for controlling automated operation of the aesthetic skin treatment unit; display the optimal set of the operating parameter to a display unit associated with the aesthetic skin treatment unit, for manually controlling the operation of the aesthetic skin treatment unit. Further, the system, wherein the processor is configured to provide the optimal set of operating parameters to the aesthetic skin treatment unit by: correcting the preset operating parameters for performing the aesthetic treatment by the aesthetic skin treatment unit, in accordance with the optimal set of operating parameters. Moreover, the system, wherein determining the optimal set of operating parameters comprises: calculating mean value of the plurality of sets of operating parameters to output optimal set of operating parameters.

In one objective of the present disclosure relates to a non-transitory computer readable medium including instructions stored thereon that when processed by at least one processor cause a system to perform operations comprising: receiving target skin data comprising at least one skin characteristic associated with skin to be treated with an aesthetic treatment by the aesthetic skin treatment unit: receiving preset operating parameters for performing the aesthetic treatment by the aesthetic skin treatment unit; analyzing the target skin data and the preset operating parameters using a plurality of trained models to predict a plurality of sets of operating parameters for the aesthetic skin treatment unit to perform the aesthetic treatment; and determining an optimal set of operating parameters for performing the aesthetic treatment by the using the aesthetic skin treatment unit, using the plurality of sets of operating parameters.

In a final objective, a non-transitory computer readable medium including instructions stored thereon that when processed by at least one processor cause a system to perform the methods of the method objectives above

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The accompanying drawings, which are incorporated in and constitute a part of this disclosure, illustrate exemplary embodiments and, together with the description, serve to explain the disclosed principles. In the figures, the left-most digit(s) of a reference number identifies the figure in which the reference number first appears. The same numbers are used throughout the figures to reference like features and components. Some embodiments of system and/or methods in accordance with embodiments of the present subject matter are now described, by way of example only, and regarding the accompanying figures, in which:

FIG. 1 illustrates an exemplary environment with a system for determining an optimal set of operating parameters for an aesthetic skin treatment unit, in accordance with some embodiments of the present disclosure;

FIG. 2 shows a detailed block diagram of system for determining an optimal set of operating parameters for an aesthetic skin treatment unit, in accordance with some embodiments of the present disclosure;

FIG. 3a shows an exemplary representation of skin characteristics received from a target skin tissue used for determining and optimal set of operating parameters for an aesthetic skin treatment unit, in accordance with some embodiments of the present disclosure;

FIG. 3b illustrates structure of network implemented for determining an optimal set of operating parameters for an aesthetic skin treatment unit, in accordance with some embodiments of the present disclosure;

FIG. 3c illustrates an exemplary schematic diagram of a backbone network using a Convolutional Neural Network (CNN) for determining an optimal set of operating parameters for an aesthetic skin treatment unit, in accordance with some embodiments of the present disclosure;

FIG. 3d illustrates structure of deep learning classifier for determining an optimal set of operating parameters for an aesthetic skin treatment unit, in accordance with some embodiments of the present disclosure;

FIG. 3e illustrates structure of autoencoder for determining an optimal set of operating parameters for an aesthetic skin treatment unit, in accordance with some embodiments of the present disclosure;

FIG. 3f illustrates structure of modified autoencoder for determining an optimal set of operating parameters for an aesthetic skin treatment unit, in accordance with some embodiments of the present disclosure;

FIG. 3g shows is a sequence diagram illustrating training of one or more training models for determining an optimal set of operating parameters for an aesthetic skin treatment unit, in accordance with some embodiments of the present disclosure;

FIG. 3h shows is a sequence diagram illustrating real-time determination of optimal set of operating parameters for an aesthetic skin treatment unit, in accordance with some embodiments of the present disclosure;

FIG. 3i illustrates a schematic representation of human skin using various filters, depicting effects of before treatment and after treatment on the skin, in accordance with some embodiments of the present disclosure;

FIG. 4 is a flow depicting a method for determining an optimal set of operating parameters for an aesthetic skin treatment unit, in accordance with some embodiments of the present disclosure; and

FIG. 5 illustrates a block diagram of an exemplary computer system for implementing embodiments consistent with the present disclosure.

The figures depict embodiments of the disclosure for purposes of illustration only. One skilled in the art will readily recognize from the following description that alternative embodiments of the structures and methods illustrated herein may be employed without departing from the principles of the disclosure described herein.

### DETAILED DESCRIPTION

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiment thereof has been shown by way of example in the drawings and will be described in detail below. It should be understood, however that it is not intended to limit the disclosure to the forms disclosed, but on the contrary, the disclosure is to cover all modifications, equivalents, and alternative falling within the spirit and the scope of the disclosure.

The terms “comprises”, “comprising”, or any other variations thereof, are intended to cover a non-exclusive inclusion, such that a setup, device, or method that comprises a list of components or steps does not include only those components or steps but may include other components or steps not expressly listed or inherent to such setup or device or method. In other words, one or more elements in a system or apparatus proceeded by “comprises . . . a” does not, without more constraints, preclude the existence of other elements or additional elements in the system or method.

The terms “includes”, “including”, or any other variations thereof, are intended to cover a non-exclusive inclusion, such that a setup, device, or method that includes a list of components or steps does not include only those components or steps but may include other components or steps not expressly listed or inherent to such setup or device or method. In other words, one or more elements in a system or apparatus proceeded by “includes . . . a” does not, without more constraints, preclude the existence of other elements or additional elements in the system or method.

The terms “comprising”, “having” and variations thereof mean “including but not limited to”, unless expressly specified otherwise.

In the following detailed description of the embodiments of the disclosure, reference is made to the accompanying drawings that form a part hereof, and in which are shown by way of illustration specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense.

Present disclosure relates to method and system for determining an optimal set of operating parameters for an aesthetic skin treatment unit. The present disclosure proposes to automate the process of determining the optimal set of operating parameters using one or more trained models. The one or more trained models are trained with a huge set of parameters related to the treatment, and preset characteristics and parameters, to output optimal operating parameters. For example, the operating parameters for laser-based system may include, but are not limited to, wavelength, spot size, fluence, pulse duration, pulse rate, pulse repetition rate.

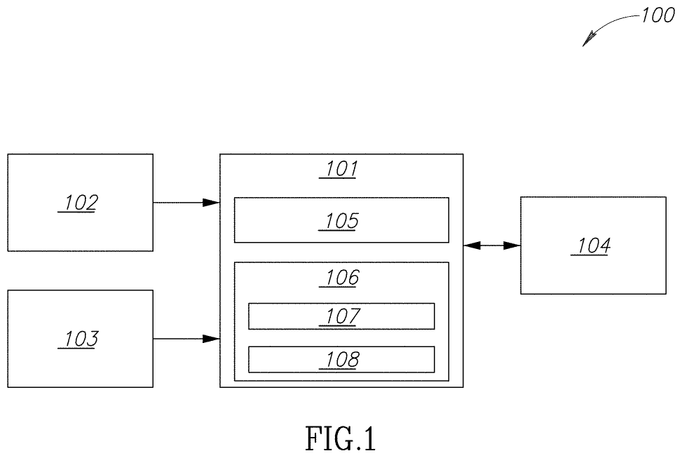

FIG. 1 illustrates an exemplary environment 100 with a system 101 for determining an optimal set of operating parameters for an aesthetic skin treatment unit 103, in accordance with some embodiments of the present disclosure. The exemplary environment 100 comprises a target skin tissue obtain data unit, hereinafter “skin data unit” 102, the system 101 and the aesthetic skin treatment unit 103. In some embodiments of the exemplary environment, the system 101 is configured to determine the optimal set of operating parameters using target skin data received from the skin data unit 102. In some embodiments, target skin data is skin characteristics or at least one skin characteristic obtained from a target skin area of skin of a person receiving treatment. Skin characteristic(s) as used herein is feature or quality belonging to skin tissue, such as, but not limited to melanin, an anatomical location, spatial and depth distribution (epidermal/dermal) of melanin, spatial and depth distribution (epidermal/dermal) of blood, melanin morphology, blood morphology, veins (capillaries) network morphology diameter and depth, spatial and depth distribution (epidermal/dermal) of collagen, water content, melanin/blood spatial homogeneity.

The system 101 may include one or more processors 104, Input/Output (I/O) interface 105 and a memory 106. In some embodiments, the I/O interface is coupled to a display for output to a user. In some embodiments, the memory 106 may be communicatively coupled to the one or more processors 104. The memory 106 stores instructions, executable by the one or more processors 104, which on execution, may cause the system 101 to determine the optimal set of operating parameters as proposed in the present disclosure. In some embodiments, the memory 106 may include one or more modules 107 and one or more collections of data 108. In some embodiments, the one or more modules 107 may be configured to perform the steps of the present disclosure using the data 108, to determine the optimal set of operating parameters. In some embodiments, each of the one or more modules 107 may be a hardware unit which may be outside the memory 106 and coupled with the system 101. In some embodiments, each of the one or more modules 107 may be one or more instructions stored in the memory 106. Such instructions may be executed by the processor 104 to perform the steps of the proposed method. In some embodiments, the system 101 may be implemented in a variety of computing systems, such as a laptop computer, a desktop computer, a Personal Computer (PC), a notebook, a smartphone, a tablet, e-book readers, a server, a network server, cloud server and the like.

In some embodiments, the aesthetic skin treatment unit 103 may be a handheld device configured to perform the aesthetic treatment on a patient. In some embodiments, the aesthetic skin treatment unit 103 is a console with a handheld component device, wherein the handheld component device is configured to be connected to the console and the combination is configured to perform the aesthetic treatment. In some embodiments, the aesthetic skin treatment unit 103 may be an energy-based unit configured to output laser beams. In some embodiments, the aesthetic skin treatment unit 103 may be used for treating skin tissue with one or more light sources.

In some embodiments, the aesthetic skin treatment unit 103 is associated with a source of treatment light along a main optical axis of the aesthetic skin treatment unit 103. In some embodiments, the aesthetic skin treatment unit 103 may include an applicator which comprises a handheld pathway for the source of treatment light, one or more sources of illumination light surrounding the main optical axis, and one or more sensors configured to obtain measured light or images along the main optical axis from a target skin tissue. In some embodiments, the measured light is reflected and backscattered light from the target skin tissue. In some embodiments, the one or more sensors configured to obtain the measured light are located in an offset optical axis from the main optical axis. In some embodiments, a correction of the offset axis sensors is accomplished using optical element angles of the one or more sensors, an algorithm or any combination thereof.

In some embodiments, the aesthetic skin treatment unit 103 may further comprise a display unit configured to display the optimal set of operating parameters provided by the system 101. In some embodiments, the optimal set of operating parameters is used by an automatic robotic energy-based system to provide optimal treatment. In some embodiments, the aesthetic skin treatment unit 103 may be at least one of, but not limited to, a manual user energy-based system or an automatic robotic energy-based system. In some embodiments, skin treatment unit 103 may be configured to utilize, at least one of, but not limited to; a laser, a lamp, LEDs or other type of light sources, a radio-frequency elements, an ultrasound elements, a microwave elements, a magnetic element, a cooling element or any combination thereof. Such aesthetic skin treatment unit 103 may be configured to provide various aesthetic treatments such as for example, hair removal, tattoo removal, skin tightening, skin rejuvenation, pigmented or vascular stain treatment, fractional aesthetic treatment, fat removal, cellulite treatments, heating, coagulating, ablating, cooling and so on.

In some embodiments, the aesthetic skin treatment unit 103 may also include a movable arm, a tool to monitor treatment process, cameras, illumination modules, sensors, spectral analyzers for backscattered light, polarizers, filters, and a controller. In some embodiments, the controller configured to activate the one or more sources of treatment light with the optimal set of operating parameters and direct the treatment light towards a target skin tissue. The one or more sensors may be configured receive images or measured light from an area of the target skin tissue, before, during, and after the treatment and provide the images or the measured light to the controller. The controller of the aesthetic skin treatment unit 103 may be configured to processes the images or the measured light received from output of the one or more sensors, define optimal treatment parameters and predict the progress of the regimen of the aesthetic treatment.

In some embodiments, operating parameters for the aesthetic skin treatment unit 103 may include parameters which define projection of light on the target skin tissue. The operating parameters may be light parameters like laser parameters, lamp parameters or any other energy parameters which may define the energy characteristic as emitted, delivered or interact with a target skin tissue by any energy modality as define above, and so on. The operating parameters may include, but are not limited to, wavelengths, spot sizes, fluences, pulse durations, pulse rates, pulse delay, number of pulses, pulse shape, repletion rate, peak power, frequency, direction, location, temperature, and so on.

Determining the optimal set of operating parameters is essential to perform effective aesthetic treatment on the target skin tissue.

In some embodiments, the system 101 may be integral part of the aesthetic skin treatment unit 103. In some embodiments, the system 101 may be externally coupled with the aesthetic skin treatment unit 103. In such embodiment, the system 101 may communicate with the aesthetic skin treatment unit 103 via a communication network. In some embodiments, the communication network may include, without limitation, a direct interconnection, Local Area Network (LAN), Wide Area Network (WAN), wireless network (e.g., using Wireless Application Protocol), the Internet, etc.

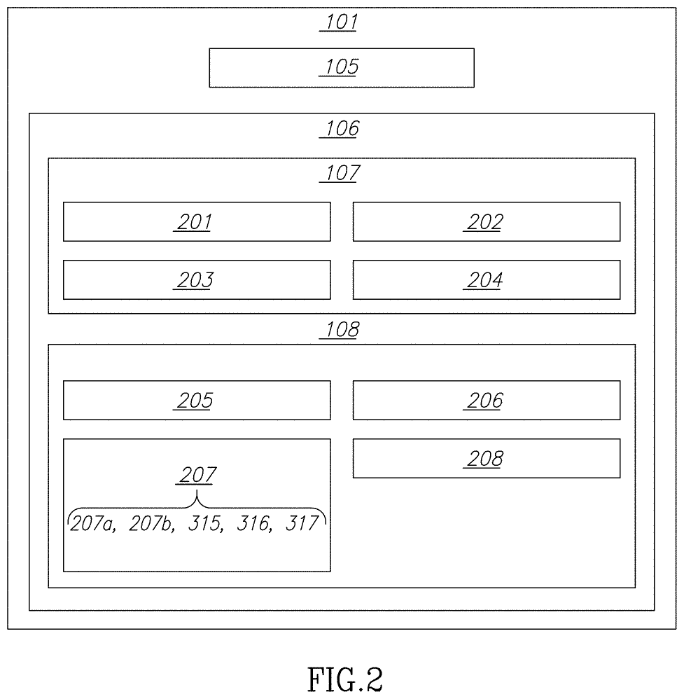

FIG. 2 shows a detailed block diagram of the system 101 for determining the optimal set of operating parameters, in accordance with some embodiments of the present disclosure. The data 108 and the one or more modules 107 in the memory 106 of the system 101 are described herein in detail.

In some embodiments, the one or more modules 107 may include, but are not limited to, a target skin data receive module 201, a treatment data analyze module 202, an operating parameter determine module 203, and one or more other modules 204, associated with the system 101. In some embodiments, the target skin data receive module receives target skin data of the skin being analyzed or analyzed for treatment. In some embodiments, the treatment data analyze module is used to analyze, parse and train the system with training data. In some embodiments, the training data comprises, at least one of the following; preset parameters of the aesthetic skin treatment unit, data related to predetermined historic (previous) failed treatments, data related to predetermined historic (previous) successful treatments, and any combination thereof.

In some embodiments, the data 108 in the memory 106 may include target skin data 205, training data 206, operating parameters data 207, and other data 208 associated with the system 101. In some embodiments, operating parameters data 207 includes at least one of, and not limited to; preset or default operating parameters 208a of the aesthetic skin treatment unit 103, three training operating parameters discussed below 315, 316, 317, optimal operating parameters 207b, or any combination thereof. In some embodiments, the preset operating parameters 207a comprises, but are not limited to; the aesthetic skin treatment unit's technical specification limits, a safety parameter as a function of the intended treatment and/or clinical effect for a specific skin type of a patient, or any combination thereof.

In some embodiments, the modules 107 and data 108 are configured such that the modules gathered and/or processed data results are then stored as part of data 108, such as training data 206 or operating parameters data 207. In some embodiments, the data 108 in the memory 106 may be processed by the one or more modules 107 of the system 101. In some embodiments, the one or more modules 107 may be implemented as dedicated units and when implemented in such a manner, the modules may be configured with the functionality defined in the present disclosure to result in a novel hardware device. As used herein, the term module may refer to an Application Specific Integrated Circuit (ASIC), an electronic circuit, a Field-Programmable Gate Arrays (FPGA), Programmable System-on-Chip (PSoC), a combinational logic circuit, and/or other suitable components that provide the described functionality.

In some embodiments, the one or more modules 107 of the present disclosure function to determine the optimal set of operating parameters 207b for the aesthetic skin treatment unit 103. The one or more modules 107 along with the data 108, may be implemented in any system, for determining the optimal set of operating parameters 207b.

The target skin data receive module 201 of the system 101 may be configured to receive the target skin data 205 from the target skin data unit 102. The target skin data 205, in some embodiments, includes skin characteristics or at least one characteristic of the target skin tissue to be treated and preset operating parameters 207a. In some embodiments, the target skin data comprises at least one pre-treatment skin characteristic (pre-treatment skin data), and at least one real-time skin characteristic (real-time skin data). The pre-treatment skin data may be characteristics associated with the skin before performed aesthetic treatment on the skin. The real-time skin data may be characteristics which are obtained in response to real-time aesthetic treatment. In some embodiments, the real-time skin data may be obtained during the aesthetic treatment, at regular intervals of time, after the aesthetic treatment or any combination thereof.

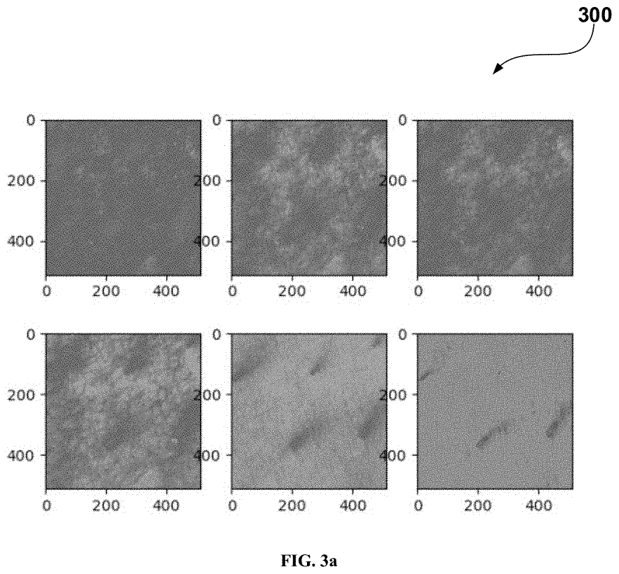

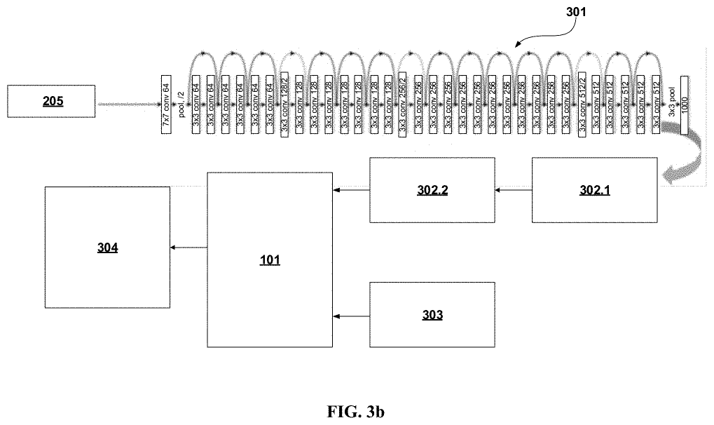

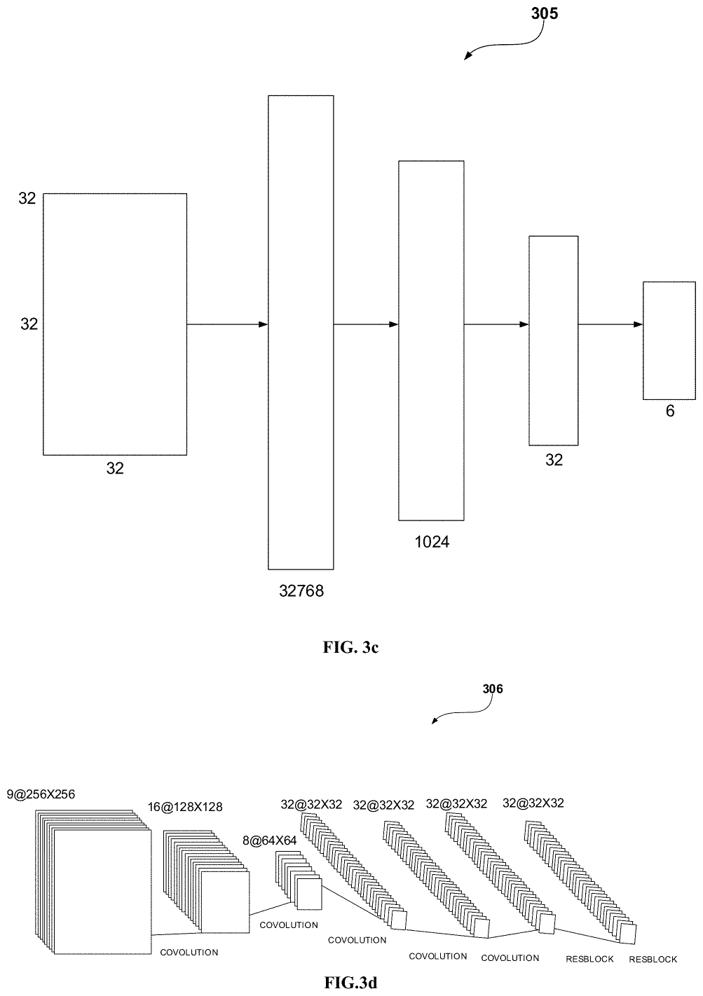

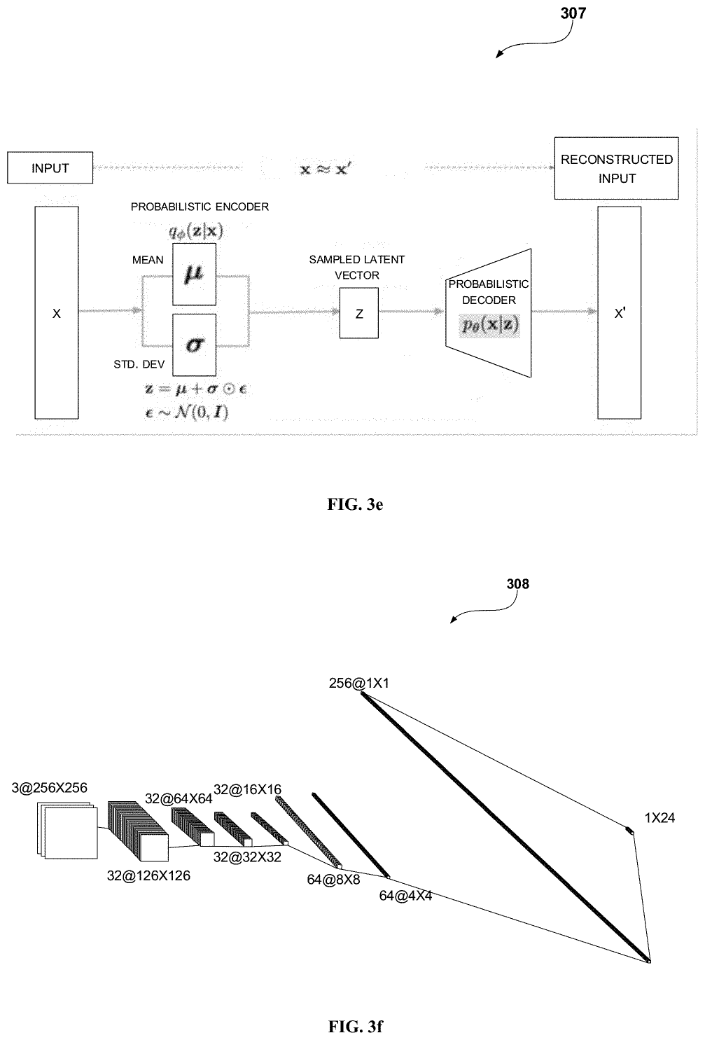

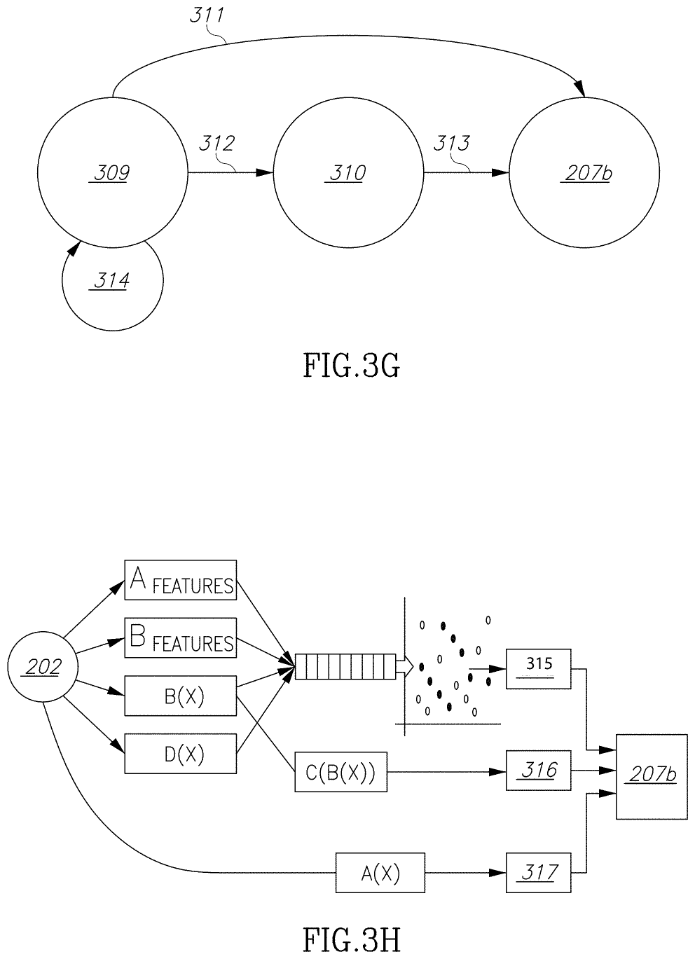

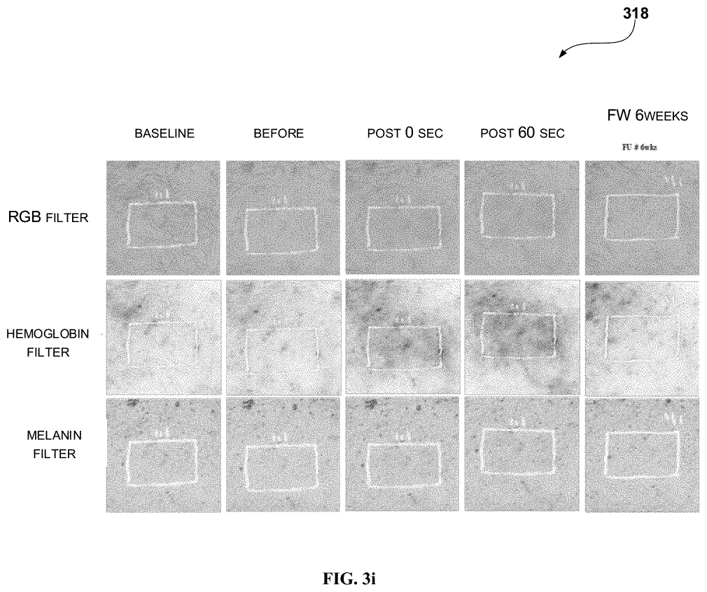

In some embodiments, the skin treatment data may be received in a form of at least one of multi-spectral images of the skin tissue, color images also known as Red Green Blue images (RGB) or a combination of both images of the skin tissue. The combination of the three channels (RGB) into a single image usually achieves a natural look of an image captured. In some embodiments, the multi-spectral images of the skin are multi-layer spatial images obtained by illuminating the skin tissue with light of one or more wavelengths as seen as an exemplary representation in 300 of FIG. 3a. Each substance in the skin tissue responds in a unique manner to light of different wavelengths. Part of the light is absorbed within the skin tissue and part of the light is reflected back from surface of the skin tissue. When investigating the skin tissue with unknown substances, and for observing features of such substances, both reflected and absorbed light of unique wavelengths may be used. By way of specific example, an amount or distribution of melanin in the skin tissue is estimated using the spectral images and in some embodiments is an important feature in determining the optimal set of operating parameters 207b.

In some embodiments, extensive information regarding the target skin tissue is obtained using different combinations of reflection coefficients in different wavelengths. In some embodiments, the multi spectral images may be captured by designing a camera associated with the aesthetic skin treatment unit 103, with special filters. Each of the special filters may be associated with required bandwidth in order to create a set of spectral images. In some embodiments, the multi spectral images may be captured by triggering the illumination light to various wavelengths using a monochrome sensor associated with the aesthetic skin treatment unit 103. In some embodiments, the multi-spectral images are used instead of the RGB image. The extensive information obtained with spectral images relates to the actual substance of the target skin tissue and its spectral behavior. When inserting such extensive information into a well-designed neural network, it is expected that the network will learn features that have to do with the target skin tissue. By way of example, skin characteristics such as melanin, concentration and distribution level, erythema and so on of the skin may be learned as the features using the multi spectral images.

In some embodiments, upon receiving the target skin data 205, the treatment data analyze module 202 may be configured to analyze the target skin data 205 using a plurality of trained models to predict a plurality of sets of operating parameters 315, 316, 317 for the aesthetic skin treatment unit 103 to perform the aesthetic treatment. In some embodiments, the plurality of trained models may include a first model (also referred to as expert A hereafter), a second model (also referred to as expert B hereafter), a third model (also referred to as expert C hereafter) and a fourth model (also referred to as expert D hereafter). In some embodiments, each of the plurality of trained models may be pre-trained using the training data 206.

In some embodiments, the training data 206 may include, but is not limited to; target skin data 205, indexes of skin historical characteristics that may be associated with before and after target skin data, hereinafter “index data”, pre-defined successful treatment data and pre-defined unsuccessful treatment data, related to the aesthetic treatment. In some embodiments, indexes and index data are associated with skin characteristics, and the indexes include but are not limited to melanin, an anatomical location, spatial and depth distribution (epidermal/dermal) of melanin, spatial and depth distribution (epidermal/dermal) of blood, melanin morphology, blood morphology, veins (capillaries) network morphology diameter and depth, spatial and depth distribution (epidermal/dermal) of collagen, water content, melanin/blood spatial homogeneity.

In some embodiments, the pre-defined successful treatment data comprises historic data related to pre-defined successful treatments. The pre-defined unsuccessful treatment data comprises historic data related to pre-defined unsuccessful treatments. The pre-defined unsuccessful treatment data, in some embodiments, includes treatments that caused physical harm, such as burns or other unsafe results. In some embodiments, the unsuccessful treatment data including physical harm is an adversary event for training a model.

In some embodiments, the training data 206 may include before treatment images and after treatment images. That is, for the pre-defined successful treatment data, image of the skin before the pre-defined successful treatment and images of the skin after the pre-defined successful treatment is used for training the plurality of trained models. Similarly, for the pre-defined unsuccessful treatment data, image of the skin before the pre-defined unsuccessful treatment and images of the skin after the pre-defined unsuccessful treatment is used for training the plurality of trained models. For example, consider the aesthetic treatment of hair removal. Images of skin before treatment and after treatment with more than 60% of relative hair loss may be used as the pre-defined successful data. Images of skin before treatment and after treatment with less than 40% of relative hair loss may be used as the pre-defined unsuccessful data. In some embodiments, one of the experts trains a network to predict skin responses to a preset parameter that is based on a physical model of the human skin for both efficacy and safety.

FIG. 3b illustrates structure of network implemented for determining the optimal set of operating parameters for an aesthetic skin treatment unit 103, in accordance with some embodiments of the present disclosure. The system 101 implements deep learning approach to determine the optimal set of operating parameters 207b. In some embodiments, the network 301 may be Residual Network (ResNet) 18, used for learning features to determine the optimal set of operating parameters 207b. In some embodiments, the input to the network 301 is the target skin data 205. Multiple layers of convolutions layers may constitute the network 301. In real-time, when determining the optimal set of the operating parameters 207b, the network 301 may be configured to output 512×1 feature vector 302.1 which is further processed to obtain 32×1 feature vector 302.2. Along with the 32×1 feature vector 302.2, the preset operating parameters 207a are inputted to the system 101, to output the optimal set of operating parameters 207b. The preset operating parameters may be in a form of 3×1 preset vector 303. FIG. 3c illustrates an exemplary schematic diagram of a backbone network 305 using a Convolutional Neural Network (CNN) for determining the optimal set of operating parameters 207b, in accordance with some embodiments of the present disclosure. In some embodiments, output may be 12×1 labelling vector 304.

In some embodiments, the plurality of trained models (experts A, B, C and D) may be plurality of deep learning models. Each of the plurality of trained models may be trained separately and independently. The plurality of trained models is trained to predict or contribute to the prediction process of the optimal set of operating parameters. Further, each of the plurality of trained models may be a function, parametric function like CNN or non-parametric function like decision tree.

In some embodiments, the first model or the expert A may be a deep-learning classifier model which is trained using the pre-defined successful treatment data. In some embodiments, the expert A may be responsible for learning visual features related to a first operating parameters 317 and may predict one or more of the optimal working parameters. The expert A may be trained under supervision with only the pre-defined successful data from input images, so that the expert A can learn only from historical pre-defined successful treatments. By way of specific example, the input images used to train expert A may be images of “before” and “after” historical aesthetic treatment/clinical trial or exemplary skin historical characteristics data 318, as depicted in FIG. 3i. Once the expert A is trained using images of “before” and “after” the aesthetic treatment, the expert A may dynamically take decisions of the aesthetic treatment, by choosing the optimal operating parameters 207b and the system 101 may work autonomously over large treatment areas. Alternatively, in some embodiments, once the expert A is trained using images of “before” and “after” the aesthetic treatment, the expert A may dynamically display decisions of the aesthetic treatment, by choosing the optimal operating parameters 207b.

The expert A may include deep learning classifier 306 as depicted on FIG. 3d. In some embodiments, the expert A may be fully CNN, with, for example, a kernel size of 4 with stride 2 and padding 1. As an example, kernel size of 4 with stride 2 and padding 1 can be parameters of the expert A, that may be chosen based on accuracy metrics relevant to the expert A on the input images provided. The kernel size, stride and padding may be chosen based on the requirement. Further, expert A includes an activation function that can be a Rectified Linear Unit (ReLU), Further, the expert A includes residual blocks (resblock) that may be the fully CNN comprising 2 convolutional blocks (cony blocks) and the residual blocks. Further, the expert A may include classifier and the classifier can be a multi-layer perceptron as depicted in FIG. 3c, with dropout layer between the layers and the ReLU as the activation function. In an exemplary embodiment, the expert A may be trained with, for example, 6 classes or more, each representing a fluence level of 10 to 60, using a cross entropy loss and Adam optimizer for optimizing the expert A. In an exemplary embodiment, the features of length 32/1024 from the classifier (e.g. 6 classes) of the expert A may be saved for subsequent/further use. The features of the expert A may be correlated directly with the optimal set of operating parameters 207b, since expert A may be trained to minimize classification error. Accordingly, the features may be forced to be correlated with the operating parameters 315. The features may be implicitly correlated to operating parameters and may not be aware of what the features represent. The implicit features, such as the index data, may be used to create a unique representation of each treatment. Based on the training of the expert A, the expert A may take “before” images of a patient in real-time and predict one or more of the optimal working parameters for the aesthetic treatment, related to visual features of working parameters, based on the received real-time “before” images of the patient.

In some embodiments, the second model or expert B may be a regressor model which is trained using index data, the pre-defined successful treatment data, and the pre-defined unsuccessful treatment data. In an embodiment, the expert B may be a deep-learning classifier and the regressor model. The expert B may be responsible for learning visual features correlated with the at least one skin characteristic of an index such as melanin index, a hemoglobin index, an anatomical location index, spatial and depth distribution (epidermal/dermal) of melanin index, spatial and depth distribution (epidermal/dermal) of blood index, melanin morphology index, blood morphology index, veins (capillaries) network morphology diameter and depth index, spatial and depth distribution (epidermal/dermal) of collagen index, water content, melanin/blood spatial homogeneity and ratio index and so on. In some embodiments, the skin characteristics are key new indexes and will be used in part to map a skin abnormality over an area to ascertain size, orientation, placement and so on, on body of the patient.

In some embodiments, the operating parameters 315, 316 and 317 are indexes of operating parameters and may be labelled in meta-data as operating index data with the index data. For example, the indexes may be numbers or integers. Further, the expert B may try to map between images (the input data) to these parameters. The parameters may be most dominant explicit features. The expert B may be trained under supervision with all the data (i.e., the pre-defined successful data and the pre-defined unsuccessful data from the input images) using a pre-defined meta-data corresponding to the explicit features. The input images such as “before” and “after” images of the historical aesthetic treatment/clinical trial as depicted in FIG. 3i, may be used to train the expert B. Further, implicit features of the expert B may be correlated with each of the explicit features of the expert B. For example, the expert B may output 3 explicit features such as the melanin index, hemoglobin index and the anatomical location. The implicit features of the expert B may be used to create a unique representation of each treatment. The features of the expert B may be saved and used for subsequent/further use. The expert B may be similar to expert A with minor modifications of hyper-parameters such as filter sizes and number of convolutional blocks. In some embodiments, the regression using a regressor may be performed with sigmoid function and mean square error lost function. In some embodiments, in order to “calibrate” expert B parameters, so expert B can regress accurately the explicit features, the mean square error cost function for instance, ∥Sigmoid (ExpertB (InputImages))- “Normalized_Melanin_index” ∥, may be minimized.

Based on the training of the expert B, the expert B may take “before” images of the patient in real time, and predict optimal working parameters related to features that are correlated with the working parameters such as a melanin index, a hemoglobin index, and an anatomical location.

In some embodiments, the third model or the expert C may be a gradient boosting model which is trained using the pre-defined successful treatment data and the index data. Expert C may implement machine learning method for problems in regression of the expert B. The expert C may produce a prediction model in the form of an ensemble of weak prediction models, typically decision trees. More specifically, the expert C may be a XG-Boost regression model. The XG-Boost is a decision-tree-based ensemble machine learning method that uses a gradient boosting framework. Further, the expert C may be responsible for predicting the working parameters by providing inputs such as the melanin index, the hemoglobin index (also known as erythema index) and the anatomical location from the expert B. The expert C may be trained under supervision with only the pre-defined successful data from the input images to train the expert C from historical success treatments. The explicit features such as the melanin index, the hemoglobin index, and the anatomical location may be the most statistically correlated parameters to the working parameters. Hence, the explicit features of the expert C may allow fluence prediction with appropriate accuracy. Based on training of the expert C, the expert C may predict the working parameters by taking inputs such as the melanin index, the hemoglobin index (also known as erythema index) and the anatomical location, from the expert B.

In some embodiments, the fourth model or expert D may be an autoencoder model, which is trained using the index data. FIG. 3e illustrates an exemplary architecture of Beta (β) Variational Auto-Encoder (BetaVAE) 307 used in the expert D for determining the optimal set of the operating parameters 207b. In an embodiment, the expert D is a Variational Auto Encoder (VAE) such as a Beta VAE. The expert D may be empirically proven to create disentangled representation or encoded representation of input images. The disentangled representation of the input images may allow to create implicit features that may be correlated with explicit visual features. The most relevant disentangled representation may be a vector, and each element in the vector may be completely correlated with a unique visual feature of the explicit visual features in the image such as hair shafts color or skin tone. The expert D may be responsible for learning the “before images”. In an exemplary embodiment, the input to the expert D may be a raster image of size 256×256×3 and the output of the expert D may be a vector of size 24×1. The output of the expert D may represent a unique representation, which may also be disentangled, of every image in the data. Further, from the raster image space of 256×256×3 and when each element represents RGB color, a 24×1 vector may be obtained. Each element representing the RGB color may have semantic meaning using expert D. The expert D may be trained using all the “before” and “after” images (i.e. before treatment) of i.e., melanin index, hemoglobin index, and RGB index of historical aesthetic treatment/clinical trials, as depicted in FIG. 3i.

The expert D may also eliminate noise and may reduce chances to overfit. In an exemplary embodiment, overfit may relate to situations where mapping of bad features may be performed for target variables (i.e., optimal set of the operating parameter 207b). For instance, if the data was considered for under specific luminance conditions, under florescent lamp for instance, then the florescent lamp may contribute to the fluence magnitude of the laser platform and may not need to learn the bad features, thus reducing chances to overfit. Since only the dominant implicit features that fully represent the image may be learned and the risk of learning a sophisticated function that maps background lighting or image compression artifacts into optimal working parameter, that is minimized using only the dominant features of the image. The expert D may be composed both from an encoder model and a decoder model of the Beta-VAE. The encoder may map the image to a 24×1 feature space vector, while the decoder may map the 24×1 feature space vector into a 256×256×3 image space matrix.

As depicted in FIG. 3e, a ‘Z’ may be the “sampled latent vector” such as the 24×1 feature space vector used to represent the image. More specifically, ‘Z’ may be a reparameterization of the data distribution mean and standard deviation (std) vectors in data distribution space using a normal distributed parameter as epsilon. The reconstructed ‘X″’ may be a nearly similar image to the input image. Based on training the expert D, the expert D takes “before” images of the patient in real-time, comprising the index data to determine the optimal set of operating parameters.

FIG. 3f illustrates a schematic diagram of modified BetaVAE encoder 308, in accordance with some embodiments of the present disclosure. The Beta variational autoencoder 308 (Beta-VAE) of the expert D may be altered empirically to suit the aesthetic skin treatment task, as depicted in FIG. 3d. The encoder may be composed from strided convolutions (such as, for example, with kernel 4, stride 2, padding 1) with ReLU activation function. The BetaVAE encoder model may be trained without supervision using a mean square error between the input and a reconstructed input. Further, a Kullback-Leibler (KL) divergence between normal distribution of mean ‘0’ and variance ‘1’ may be used to the data distribution mean and the std vector. Further, the expert D may also perform infinite generation of data and the data augmentation. Since the expert D has modelled the distribution of the data, the expert D may be sampled from the distribution of the data by explicitly altering the ‘Z’ vector. The expert D sampled with latent vector may be saved for subsequent use.

FIG. 3g is a sequence diagram of training procedure of the plurality of trained models, in accordance with some embodiments of the present disclosure. The expert A 311, the expert B 312, the expert C 313 and the expert D 314 may be trained as depicted in FIG. 3g, and the training may be locally (i.e. offline) performed using skin data 309 that is associated with target skin data 205, and the index data 310. The training may be performed to train each of the expert A 311, the expert B 312, the expert C 313 and the expert D 314, to be efficient at specific task of prediction of the optimal set of operating parameters. The expert A 311, the expert B 312, the expert C 313 and the expert D 314 may be trained separately.

The expert A 311 may be trained under supervision using the “before” and “after” images of the skin index data 310 and the pre-defined successful treatment data and corresponding preset operating parameters. The expert B 312 may be trained under supervision using the “before” and “after” images of the index data 310 of all of the historical aesthetic treatments (i.e., both the pre-defined successful treatment data and pre-defined unsuccessful treatment data), and corresponding recorded meta-parameters such as the index data. The expert C 313 may be trained under supervision using the index data of both the pre-defined successful treatment data and pre-defined unsuccessful treatment data. The expert D 314 may be trained without supervision using the “before” and “after” images of the index data of all both the pre-defined successful treatment data and pre-defined unsuccessful treatment data.

FIG. 3h shows a sequence diagram illustrating real-time determination of the optimal set of operating parameters for an aesthetic skin treatment unit 103. In some embodiments, the plurality of operating parameters may be a first set of operating parameters 317, second set of operating parameters 316 and third set of operating parameters 315. The treatment data analyze module 202 may be configured to analyze the target skin data 205 to obtain the plurality sets of operating parameters. The first set of operating parameters may be obtained by analyzing the target skin data 205 using the first model. Route to obtain the first set of operating parameters may be referred to as deep end2end route. The target skin data 205 is provided as input to the expert A and the output of the expert A is the first set of operating parameters. Analyses to obtain the first set of operating parameters includes classifying the skin characteristics to identify one or more first classes for the skin characteristics and correlate the one or more first classes with the preset operating parameters, to obtain the first set of operating parameters.

The second set of operating parameters may be obtained by analyzing the target skin data 205 using the second model and the third model. Route for determining the second set of operating parameters may be referred to as extreme decision tree route. The target skin data 205 is providing as input to the expert B to extract predicted operating parameters. Then, the index data may be passed into expert C to predict the second set of the operating parameters. Analyses to obtain the second set of operating parameters includes to extract, using the second model, real-time skin data from the skin data 309. Further, using the third model, the real-time skin data is correlated with the preset operating parameters, to obtain the second set of operating parameters amongst the plurality of sets of operating parameters.

The third set of operating parameters 315 may be obtained by analyzing the target skin data 205 using the first model, the second model, and the fourth model. Route for determining the third set of operating parameters may be referred to as spatial interpolation route. In some embodiments, analyses to obtain the second set of operating parameters includes to receive the one or more first classes from the first model and receive the real-time skin data and one or more second classes obtained by classifying the real-time skin data, from the second model. Further, the encoded representation is obtained from the fourth model for the skin data 309 using the index data 310. A semantic representation is generated for the target skin data 205 by concatenating the one or more first classes, the real-time skin data, the one or more second classes and the encoded representation. The semantic representation may be a single vector of size (32+32*3+3+24*3), standing for a unique semantic representation of each of the target skin data 205. In some embodiments, information in the semantic representation is interpolated to obtain a third set of operating parameters from the plurality of sets of operating parameters. The interpolation may be performed using a kriging interpolation (i.e., spatial interpolation) method with the pre-defined successful treatment data. Further, unlike simple nearest neighbor approach or distance like interpolation technique, the machine learning method may be used in the direct prediction method and the multi-dimensional interpolation problem as an optimization problem may be formulated. The kriging interpolation method is similar to inverse distance weighting interpolation, where the kriging interpolation method weighs the surrounding measured treatments to derive a prediction for an unmeasured treatment (i.e. the query). The equation for both interpolators may be formed as a weighted sum of the data:

Z(X_{?})=Σᵢ₌₁⁷²λᵢZ(Xᵢ)

Where, Z(X_{?}) is the operating parameters value at the current treatment (the unknown treatment), μᵢ is an unknown weight for the measured value at treatment I, X_{?} is a 203 features vector representing a treatment.

In the Naive Bayes Nearest Neighbor (NBNN) method, the n-closest treatments among the past treatments to the query treatment, is the best treatment to follow in terms of operating parameters. In inverse distance weighting interpolation, the weight, λᵢ may depend completely on the distance to the prediction operating parameters feature vector. However, taking into account all the green treatments unlike the naïve nearest neighbor method, in the kriging method the weights are based not only on the distance between the measured points and the prediction location but also on the overall spatial arrangement of the measured points. To use the spatial arrangement in the weights, the spatial auto correlation must be quantified. Thus, in ordinary kriging interpolation method, the weight, λᵢ, depends on a fitted model to the measured points, the distance to the prediction location, and the spatial relationships among the measured values around the prediction location. The weights are learnt from the green treatments by minimizing the kriging optimization problem. The kriging interpolation method may form weights from surrounding past treatments to predict unknown fluence of the new treatment.

The operating parameter determine module 203 may be configured to determine the optimal set of operating parameters 207b. The optimal set of the operating parameters may be determined using the plurality of sets of operating parameters. The average of the 3 routes may be the optimal working parameters to lead to the best clinical outcome. The std vector may be a measure of prediction confidence. In some embodiments, the optimal set of the operating parameters are an average of all routes as shown in below equation:

$$Optimal set of operating parameters = \frac{First set + Second set + Third set}{3}$$

In some embodiments, confidence level of the determination may be derived by calculating the standard deviation for the determined optimal set of operating parameters.

In some embodiments, the optimal set of operating parameters determined by the system 101 may be provided to the aesthetic skin treatment unit 103, for controlling automated operation of the aesthetic skin treatment unit 103. In some embodiments, by providing the optimal set of operating parameters, the preset operating parameters 207a may be corrected for performing the aesthetic treatment by the aesthetic skin treatment unit 103, in accordance with the optimal set of operating parameters. In some embodiments, the optimal set of operating parameters determined by the system 101 may be displayed on the display unit associated with the aesthetic skin treatment unit 103. Using the displayed optimal set of operating parameters, the user may manually control the operation of the aesthetic skin treatment unit 103. In some embodiments, each of the plurality of operating parameters and the optimal set of operating parameters is stored in the memory 106 as the operating parameters data 207.

Further, the system 101 may be configured to perform quantification of pigmented and vascular content in various lesions in laser therapy, such as photoaging, epidermal pigmentation, lentigines (age spots, sunspots), facial rosacea port wine stains and so on. The quantification for determining the optimal set of operating parameters may be envisioned to be three-dimensional (spatial and depth) melanin and blood content segmentation and quantification. In some embodiments, the system 101 may be configured to perform quantification of immediate response. This will allow prediction of the treatment outcome (that might take months) and real time treatment parameters adjustment. In some embodiments, the system 101 may be configured to perform quantification of scars (using collagen blood and melanin) morphology and depth for optimal (CO2 fractional) treatment settings. In some embodiments, the system 101 may be configured to perform segmentation and quantification of vascular networks in terms of morphology, depth, and diameters for localizing the best spatial position for treatment (closing the main valve might be an optimal solution for superficial vein treatment). In some embodiments, the system 101 may be configured to perform tattoo absorption spectrum determination for correct treatment wavelength selection. In some embodiments, the system 101 may be configured to perform quantification of skin water content and corresponding hydration condition. In some embodiments, the system 101 may be configured to perform estimation of skin aging followed by treatment recommendations. In some embodiments, the system 101 may be configured to perform tanning level quantification.

In some embodiments, the system 101 may receive data for determining the optimal set of operating parameters via the I/O interface 105. The received data may include, but is not limited to, at least one of the treatment data, and so on. Also, the system 101 may transmit data for determining the optimal set of operating parameters via the I/O interface 105. The transmitted data may include, but is not limited to, the optimal set of operating parameters, output of each of the plurality of trained models, and so on.

The other data 208 may store data, including temporary data and temporary files, generated by modules for performing the various functions of the system 101. The one or more modules 107 may also include other modules 204 to perform various miscellaneous functionalities of the system 101. It will be appreciated that such modules may be represented as a single module or a combination of different modules.

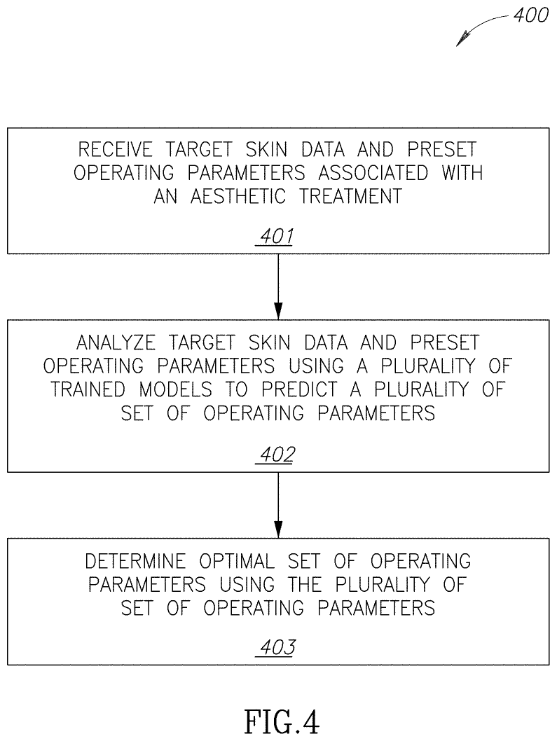

FIG. 4 is a flow chart depicting a method 400 for determining the optimal set of operating parameters

At block 401, the system 101 is configured to receive the target skin data 205 comprising at least one skin characteristics associated with the target skin tissue to be treated with the aesthetic treatment, and the preset operating parameters 207a for performing the aesthetic treatment. The target skin data comprises at least one of the pre-treatment skin data, and the real-time skin data in response to the aesthetic treatment. In some embodiments, the skin data 205 is received in a form of at least one of multi-spectral images of the target skin, RGB images of the target skin, or any combination thereof.

At block 402, the system 101 is configured to analyze the target skin data 205 using the plurality of trained models to predict the plurality of sets of operating parameters for the aesthetic skin treatment unit 103 to perform the aesthetic treatment. The plurality of trained models comprises the first model, the second model, the third model and the fourth model. Each of the plurality of trained models is pre-trained using index data, the pre-defined successful treatment data and the pre-defined unsuccessful treatment data, related to the aesthetic treatment.

In some embodiments, the first model is the deep-learning classifier model trained using the pre-defined successful treatment data. In some embodiments, the second model is the regressor model trained using the index data, the pre-defined successful treatment data, and the pre-defined unsuccessful treatment data. In some embodiments, the third model is the gradient boosting model trained using the pre-defined successful treatment data and the index data. In an embodiment, the fourth model is the autoencoder model trained using the index data. In some embodiments, analyzing the target skin data 205 using the first model from the plurality of trained models includes to classify the skin characteristics to identify one or more first classes for the skin characteristics and correlate the one or more first classes with the preset operating parameters, to obtain first set of operating parameters amongst the plurality of sets of operating parameters.

In some embodiments, analyzing the target skin data 205 using the second model and the third model from the one or more trained models includes to extract, using the second model, the real-time skin data from the skin data 309 and correlate, using the third model, the real-time skin data with the preset operating parameters, to obtain the second set of operating parameters amongst the plurality of sets of operating parameters. In some embodiments, analyzing the treatment data using the first model, the second model and the third model from the one or more trained models includes to receive the one or more first classes from the first model and the real-time skin data and the one or more second classes obtained by classifying the real-time skin data, from the second model. Further, the encoded representation for the skin data 309, using the index data 310 is generated. A semantic representation for the target skin data 205 is generated by concatenating the one or more first classes, the real-time skin data, the one or more second classes and the encoded representation. Information in the semantic representation is interpolated to obtain the third set of operating parameters from the plurality of sets of operating parameters.

At block 403, the system 101 is configured to determine the optimal set of operating parameters for performing the aesthetic treatment by the using the aesthetic skin treatment unit 103, using the plurality of sets of operating parameters. In some embodiments, mean value of the plurality of sets of operating parameters is calculated to output optimal set of operating parameters.

In some embodiments, once trained the system will receive target skin data 205 from the skin data unit 102 and the treatment data analyze module 202 will analyze the target skin data, the safety parameters of the treatment, the aesthetic skin treatment unit technical limits using a plurality of trained models to determine the optimal treatment parameters.

As illustrated in FIG. 4, the method 400 may include one or more blocks for executing processes in the system 101. The method 400 may be described in the general context of computer executable instructions. Generally, computer executable instructions can include routines, programs, objects, components, data structures, procedures, modules, and functions, which perform particular functions or implement particular abstract data types.

The order in which the method 400 is described may not intended to be construed as a limitation, and any number of the described method blocks can be combined in any order to implement the method. Additionally, individual blocks may be deleted from the methods without departing from the scope of the subject matter described herein. Furthermore, the method can be implemented in any suitable hardware, software, firmware, or combination thereof.

Embodiments herein determine key operating parameters for optimal clinical outcomes based on spectral image data. Embodiments herein may determine the optimal working parameters before the initial treatment and during treatment by observing and analyzing the immediate response and adjusting the operating parameters as required.

Embodiments herein may help in determining the optimal operating parameters without manually considering the skin attributes such as skin type, presence of tanning, hair color, hair density, vessel diameter and depth, lesion type, pigment depth, pigment intensity, tattoo color and type to decide which operating parameters to use such as wavelength, spot size, fluence, pulse duration, pulse rate. Embodiments herein may eliminate trial-and-error method of aesthetic treatment or observing the immediate responses and avoiding fine tune the working parameters accordingly.

Computing System

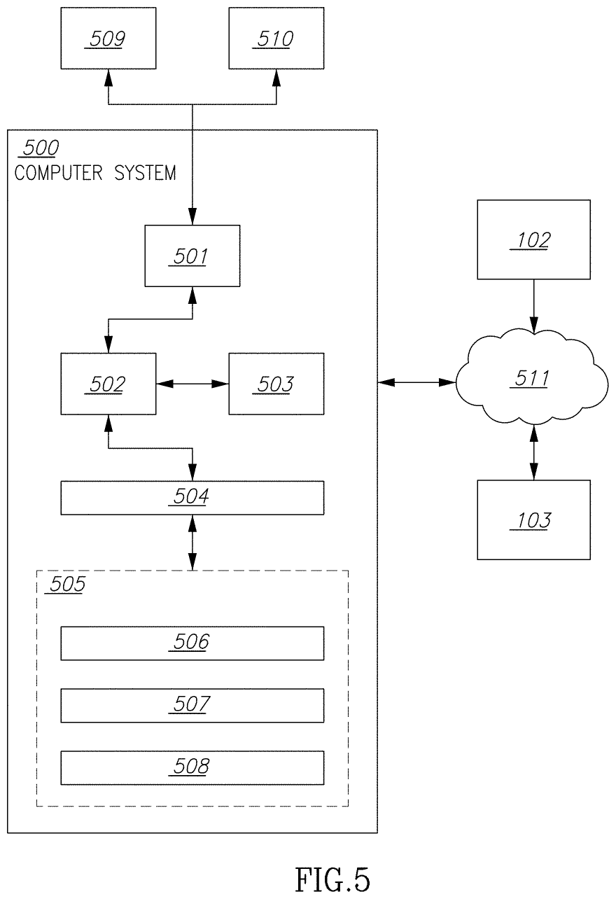

FIG. 5 illustrates a block diagram of an exemplary computer system 500 for implementing embodiments consistent with the present disclosure. In some embodiments, the computer system 500 is used to implement the system 101 for determining the optimal set of operating parameters. The computer system 500 may include a central processing unit (“CPU” or “processor”) 502. The processor 502 may include at least one data processor for executing processes in Virtual Storage Area Network. The processor 502 may include specialized processing units such as, integrated system (bus) controllers, memory management control units, floating point units, graphics processing units, digital signal processing units, etc.

The processor 502 may be disposed in communication with one or more input/output (I/O) devices 509 and 510 via I/O interface 501. The I/O interface 501 may employ communication protocols/methods such as, without limitation, audio, analog, digital, monaural, RCA, stereo, IEEE-1394, serial bus, universal serial bus (USB), infrared, PS/2, BNC, coaxial, component, composite, digital visual interface (DVI), high-definition multimedia interface (HDMI), radio frequency (RF) antennas, S-Video, VGA, IEEE 802.n /b/g/n/x, Bluetooth, cellular (e.g., code-division multiple access (CDMA), high-speed packet access (HSPA+), global system for mobile communications (GSM), long-term evolution (LTE), WiMax, or the like), etc.

Using the I/O interface 501, the computer system 500 may communicate with one or more I/O devices 509 and 510. For example, the input devices 509 may be an antenna, keyboard, mouse, joystick, (infrared) remote control, camera, card reader, fax machine, dongle, biometric reader, microphone, touch screen, touchpad, trackball, stylus, scanner, storage device, transceiver, video device/source, etc. The output devices 510 may be a printer, fax machine, video display (e.g., cathode ray tube (CRT), liquid crystal display (LCD), light-emitting diode (LED), plasma, Plasma Display Panel (PDP), Organic light-emitting diode display (OLED) or the like), audio speaker, etc.

In some embodiments, the computer system 500 may consist of the system 101.

The processor 502 may be disposed in communication with a communication network 511 via a network interface 503. The network interface 503 may communicate with the communication network 511. The network interface 503 may employ connection protocols including, without limitation, direct connect, Ethernet (e.g., twisted pair 10/100/1000 Base T), transmission control protocol/internet protocol (TCP/IP), token ring, IEEE 802.11a/b/g/n/x, etc. The communication network may include, without limitation, a direct interconnection, local area network (LAN), wide area network (WAN), wireless network (e.g., using Wireless Application Protocol), the Internet, etc. Using the network interface 503 and the communication network 511, the computer system 500 may communicate with at least one of a skin data unit 102 and an aesthetic skin treatment unit 103, for determining the optimal set of operating parameters. The network interface 503 may employ connection protocols include, but not limited to, direct connect, Ethernet (e.g., twisted pair 10/100/1000 Base T), transmission control protocol/internet protocol (TCP/IP), token ring, IEEE 802.11a/b/g/n/x, etc.

The communication network 511 includes, but is not limited to, a direct interconnection, an e-commerce network, a peer to peer (P2P) network, local area network (LAN), wide area network (WAN), wireless network (e.g., using Wireless Application Protocol), the Internet, Wi-Fi, and such. The first network and the second network may either be a dedicated network or a shared network, which represents an association of the different types of networks that use a variety of protocols, for example, Hypertext Transfer Protocol (HTTP), Transmission Control Protocol/Internet Protocol (TCP/IP), Wireless Application Protocol (WAP), etc., to communicate with each other. Further, the first network and the second network may include a variety of network devices, including routers, bridges, servers, computing devices, storage devices, etc.

In some embodiments, the processor 502 may be disposed in communication with a memory 505 (e.g., RAM, ROM, etc. not shown in FIG. 5) via a storage interface 504. The storage interface 504 may connect to memory 505 including, without limitation, memory drives, removable disc drives, etc., employing connection protocols such as, serial advanced technology attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etc.

The memory 505 may store a collection of programs or database components, including, without limitation, user interface 506, an operating system 507, web browser 508 etc. In some embodiments, computer system 500 may store user/application data, such as, the data, variables, records, etc., as described in this disclosure. Such databases may be implemented as fault-tolerant, relational, scalable, secure databases such as Oracle® or Sybase®.

The operating system 507 may facilitate resource management and operation of the computer system 500. Examples of operating systems include, without limitation, APPLE MACINTOSH® OS X, UNIX®, UNIX-like system distributions (E.G., BERKELEY SOFTWARE DISTRIBUTION™ (BSD), FREEBSD™, NETBSD™, OPENBSD™, etc.), LINUX DISTRIBUTIONS™ (E.G., RED HAT™, UBUNTU™, KUBUNTU™, etc.), IBM™ OS/2, MICROSOFT™ WINDOWS™ (XP™, VISTA™/7/8, 10 etc.), APPLE® IOS™, GOOGLE® ANDROID™, BLACKBERRY® OS, or the like.

In some embodiments, the computer system 500 may implement a web browser 508 stored program component. The web browser 508 may be a hypertext viewing application, such as Microsoft Internet Explorer, Google Chrome, Mozilla Firefox, Apple Safari, etc. Secure web browsing may be provided using Hypertext Transport Protocol Secure (HTTPS), Secure Sockets Layer (SSL), Transport Layer Security (TLS), etc. Web browsers 508 may utilize facilities such as AJAX, DHTML, Adobe Flash, JavaScript, Java, Application Programming Interfaces (APIs), etc. In some embodiments, the computer system 500 may implement a mail server stored program component. The mail server may be an Internet mail server such as Microsoft Exchange, or the like. The mail server may utilize facilities such as ASP, ActiveX, ANSI C++/C#, Microsoft .NET, Common Gateway Interface (CGI) scripts, Java, JavaScript, PERL, PHP, Python, WebObjects, etc. The mail server may utilize communication protocols such as Internet Message Access Protocol (IMAP), Messaging Application Programming Interface (MAPI), Microsoft Exchange, Post Office Protocol (POP), Simple Mail Transfer Protocol (SMTP), or the like. In some embodiments, the computer system 500 may implement a mail client stored program component. The mail client may be a mail viewing application, such as Apple Mail, Microsoft Entourage, Microsoft Outlook, Mozilla Thunderbird, etc.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include Random Access Memory (RAM), Read-Only Memory (ROM), volatile memory, non-volatile memory, hard drives, Compact Disc (CD) ROMs, DVDs, flash drives, disks, and any other known physical storage media.

The described operations may be implemented as a method, system or article of manufacture using standard programming and/or engineering techniques to produce software, firmware, hardware, or any combination thereof. The described operations may be implemented as code maintained in a “non-transitory computer readable medium”, where a processor may read and execute the code from the computer readable medium. The processor is at least one of a microprocessor and a processor capable of processing and executing the queries. A non-transitory computer readable medium may include media such as magnetic storage medium (e.g., hard disk drives, floppy disks, tape, etc.), optical storage (CD-ROMs, DVDs, optical disks, etc.), volatile and non-volatile memory devices (e.g., EEPROMs, ROMs, PROMs, RAMs, DRAMs, SRAMs, Flash Memory, firmware, programmable logic, etc.), etc. Further, non-transitory computer-readable media may include all computer-readable media except for a transitory. The code implementing the described operations may further be implemented in hardware logic (e.g., an integrated circuit chip, Programmable Gate Array (PGA), Application Specific Integrated Circuit (ASIC), etc.).

An “article of manufacture” includes non-transitory computer readable medium, and/or hardware logic, in which code may be implemented. A device in which the code implementing the described embodiments of operations is encoded may include a computer readable medium or hardware logic. Of course, those skilled in the art will recognize that many modifications may be made to this configuration without departing from the scope of the invention, and that the article of manufacture may include suitable information bearing medium known in the art.

The terms “an embodiment”, “embodiment”, “embodiments”, “the embodiment”, “the embodiments”, “one or more embodiments”, “some embodiments”, and “one embodiment” mean “one or more (but not all) embodiments of the invention(s)” unless expressly specified otherwise.

The terms “a”, “an” and “the” mean “one or more”, unless expressly specified otherwise.

A description of an embodiment with several components in communication with each other does not imply that all such components are required. On the contrary a variety of optional components are described to illustrate the wide variety of possible embodiments of the invention.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated operations of FIG. 4 shows certain events occurring in a certain order. In alternative embodiments, certain operations may be performed in a different order, modified, or removed. Moreover, steps may be added to the above-described logic and still conform to the described embodiments. Further, operations described herein may occur sequentially or certain operations may be processed in parallel. Yet further, operations may be performed by a single processing unit or by distributed processing units.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the disclosure of the embodiments of the invention is intended to be illustrative, but not limiting, of the scope of the invention, which is set forth in the following claims.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What we claim is:

1. A method for determining an optimal set of operating parameters for an aesthetic skin treatment unit, comprising:
receiving target skin data comprising at least one skin characteristic associated with skin to be treated with an aesthetic treatment by the aesthetic skin treatment unit:
receiving preset operating parameters for performing the aesthetic treatment by the aesthetic skin treatment unit;
analyzing the target skin data and the preset operating parameters using a plurality of trained models to predict a plurality of sets of operating parameters for the aesthetic skin treatment unit to perform the aesthetic treatment; and
determining an optimal set of operating parameters for performing the aesthetic treatment by the using the aesthetic skin treatment unit, using the plurality of sets of operating parameters.

2. The method of claim 1, wherein the target skin data comprises at least one of pre-treatment skin data, real-time skin data in response to the aesthetic treatment, or any combination thereof.

3. The method of claim 1, the target skin data is received in a form of at least one of multi-spectral images of the skin, Red Green Blue (RGB) images of the skin, or any combination thereof.

4. The method of claim 3, wherein the multi-spectral images of the skin are obtained by illuminating light on the skin with a plurality of wavelengths, and by analyzing the multi-spectral images obtained, the one or more trained models are configured to achieve depth analysis of the skin.

5. The method of claim 1, wherein the plurality of trained models comprises a first model, a second model, a third model and a fourth model, wherein each of the plurality of trained models are pre-trained using index data, pre-defined successful treatment data and pre-defined unsuccessful treatment data, related to the aesthetic treatment.

6. The method of claim 5, wherein the first model is a deep-learning classifier model trained using the pre-defined successful treatment data,
wherein the second model is a regressor model trained using the index data, the pre-defined successful treatment data, and the pre-defined unsuccessful treatment data,
wherein the third model is a gradient boosting model trained using the pre-defined successful treatment data and the index data, and
wherein the fourth model is an autoencoder model trained using the index data.

7. The method of claim 5, wherein analyzing the target skin data using the first model from the plurality of trained models, comprises:
classifying the at least one skin characteristic of the target skin data to identify one or more first classes for the at least one skin characteristic; and
correlating the one or more first classes with the preset operating parameters, to obtain first set of operating parameters amongst the plurality of sets of operating parameters.

8. The method of claim 7, wherein analyzing the target skin data using the second model and the third model from the one or more trained models comprises:
extracting, using the second model, real-time skin data from the skin target skin data; and
correlating, using the third model, the real-time skin data with the preset operating parameters, to obtain second set of operating parameters amongst the plurality of sets of operating parameters.

9. The method of claim 8, wherein analyzing the target skin data using the first model, the second model and the third model from the one or more trained models comprises:
receiving the one or more first classes from the first model;
receiving the real-time data and one or more second classes obtained by classifying the real-time skin data, from the second model;
generating, using the fourth model, encoded representation for the skin data using the index data;
generating semantic representation for the target skin data by concatenating the one or more first classes, the real-time skin data, the one or more second classes and the encoded representation; and
interpolating information in the semantic representation to obtain a third set of operating parameters from the plurality of sets of operating parameters.

10. The method of claim 1, further comprises one of:
providing the optimal set of operating parameters to the aesthetic skin treatment unit, for controlling automated operation of the aesthetic skin treatment unit;
displaying the optimal set of the operating parameter to a display unit associated with the aesthetic skin treatment unit, for manually controlling the operation of the aesthetic skin treatment unit.

11. The method of claim 10, wherein providing the optimal set of operating parameters to the aesthetic skin treatment unit comprises:
correcting the preset operating parameters for performing the aesthetic treatment by the aesthetic skin treatment unit, in accordance with the optimal set of operating parameters.

12. The method of claim 1, wherein determining the optimal set of operating parameters comprises:
calculating mean value of the plurality of sets of operating parameters to output optimal set of operating parameters.

13. A system for determining an optimal set of operating parameters for an aesthetic skin treatment unit, comprises:
a processor; and
a memory communicatively coupled to the processor, wherein the memory stores processor-executable instructions, which, on execution, cause the processor to:
receive target skin data comprising at least one skin characteristic associated with skin to be treated with an aesthetic treatment by the aesthetic skin treatment unit:
receive preset operating parameters for performing the aesthetic treatment by the aesthetic skin treatment unit;
analyze the target skin data and the preset operating parameters using a plurality of trained models to predict a plurality of sets of operating parameters for the aesthetic skin treatment unit to perform the aesthetic treatment; and
determine an optimal set of operating parameters for performing the aesthetic treatment by the using the aesthetic skin treatment unit, using the plurality of sets of operating parameters.

14. The system of claim 13, wherein the target skin data comprises at least one of pre-treatment skin data, real-time skin data in response to the aesthetic treatment, or any combination thereof.

15. The system of claim 13, the target skin data is received in a form of at least one of multi-spectral images of the skin, Red Green Blue (RGB) images of the skin, or any combination thereof.

16. The system of claim 15, wherein the multi-spectral images of the skin are obtained by illuminating light on the skin with a plurality of wavelengths, and by analyzing the multi-spectral images obtained, the one or more trained models are configured to achieve depth analysis of the skin.

17. The system of claim 13, wherein the plurality of trained models comprises a first model, a second model, a third model and a fourth model, wherein each of the plurality of trained models are pre-trained using index data, pre-defined successful treatment data and pre-defined unsuccessful treatment data, related to the aesthetic treatment.

18. The system of claim 17, wherein the first model is a deep-learning classifier model trained using the pre-defined successful treatment data,
wherein the second model is a regressor model trained using the index data, the pre-defined successful treatment data, and the pre-defined unsuccessful treatment data,
wherein the third model is a gradient boosting model trained using the pre-defined successful treatment data and the index data, and
wherein the fourth model is an autoencoder model trained using the index data.

19. The system of claim 17, wherein the processor is configured to analyze the target skin data using the first model from the plurality of trained models by:
classifying the at least one skin characteristic of the target skin data to identify one or more first classes for the at least one skin characteristic; and
correlating the one or more first classes with the preset operating parameters, to obtain first set of operating parameters amongst the plurality of sets of operating parameters.

20. The system of claim 17, wherein the processor is configured to analyze the target skin data using the second model and the third model from the one or more trained models by:
extracting, using the second model, real-time skin data from the skin target skin data; and
correlating, using the third model, the real-time skin data with the preset operating parameters, to obtain second set of operating parameters amongst the plurality of sets of operating parameters.

21. The system of claim 17, wherein the processor is configured to analyze the target skin data using the first model, the second model and the third model from the one or more trained models by:
receiving the one or more first classes from the first model;
receiving the real-time data and one or more second classes obtained by classifying the real-time skin data, from the second model;
generating, using the fourth model, encoded representation for the skin data using the index data;
generating semantic representation for the target skin data by concatenating the one or more first classes, the real-time skin data, the one or more second classes and the encoded representation; and
interpolating information in the semantic representation to obtain a third set of operating parameters from the plurality of sets of operating parameters.

22. The system of claim 13, further comprises the processor configured to:
provide the optimal set of operating parameters to the aesthetic skin treatment unit, for controlling automated operation of the aesthetic skin treatment unit;
display the optimal set of the operating parameter to a display unit associated with the aesthetic skin treatment unit, for manually controlling the operation of the aesthetic skin treatment unit.

23. The system of claim 13, wherein the processor is configured to provide the optimal set of operating parameters to the aesthetic skin treatment unit by:
correcting the preset operating parameters for performing the aesthetic treatment by the aesthetic skin treatment unit, in accordance with the optimal set of operating parameters.

24. The system of claim 13, wherein determining the optimal set of operating parameters comprises:
calculating mean value of the plurality of sets of operating parameters to output optimal set of operating parameters.

25. A non-transitory computer readable medium including instructions stored thereon that when processed by at least one processor cause a system to perform operations comprising:
receiving target skin data comprising at least one skin characteristic associated with skin to be treated with an aesthetic treatment by the aesthetic skin treatment unit:
receiving preset operating parameters for performing the aesthetic treatment by the aesthetic skin treatment unit;
analyzing the target skin data and the preset operating parameters using a plurality of trained models to predict a plurality of sets of operating parameters for the aesthetic skin treatment unit to perform the aesthetic treatment; and
determining an optimal set of operating parameters for performing the aesthetic treatment by the using the aesthetic skin treatment unit, using the plurality of sets of operating parameters.

26. The medium of claim 25, wherein the target skin data comprises at least one of pre-treatment skin data, real-time skin data in response to the aesthetic treatment, or any combination thereof.

27. The medium of claim 25, the target skin data is received in a form of at least one of multi-spectral images of the skin, Red Green Blue (RGB) images of the skin, or any combination thereof.

28. The medium of claim 27, wherein the multi-spectral images of the skin are obtained by illuminating light on the skin with a plurality of wavelengths, and by analyzing the multi-spectral images obtained, the one or more trained models are configured to achieve depth analysis of the skin.

29. The medium of claim 25, wherein the plurality of trained models comprises a first model, a second model, a third model and a fourth model, wherein each of the plurality of trained models are pre-trained using index data, pre-defined successful treatment data and pre-defined unsuccessful treatment data, related to the aesthetic treatment.

30. The medium of claim 29, wherein the first model is a deep-learning classifier model trained using the pre-defined successful treatment data,
wherein the second model is a regressor model trained using the index data, the pre-defined successful treatment data, and the pre-defined unsuccessful treatment data,
wherein the third model is a gradient boosting model trained using the pre-defined successful treatment data and the index data, and
wherein the fourth model is an autoencoder model trained using the index data.

31. The medium of claim 29, wherein analyzing the target skin data using the first model from the plurality of trained models, comprises:
classifying the at least one skin characteristic of the target skin data to identify one or more first classes for the at least one skin characteristic; and
correlating the one or more first classes with the preset operating parameters, to obtain first set of operating parameters amongst the plurality of sets of operating parameters.

32. The medium of claim 29, wherein analyzing the target skin data using the second model and the third model from the one or more trained models comprises:
extracting, using the second model, real-time skin data from the skin target skin data; and
correlating, using the third model, the real-time skin data with the preset operating parameters, to obtain second set of operating parameters amongst the plurality of sets of operating parameters.

33. The medium of claim 29, wherein analyzing the target skin data using the first model, the second model and the third model from the one or more trained models comprises:
receiving the one or more first classes from the first model;
receiving the real-time data and one or more second classes obtained by classifying the real-time skin data, from the second model;
generating, using the fourth model, encoded representation for the skin data using the index data;
generating semantic representation for the target skin data by concatenating the one or more first classes, the real-time skin data, the one or more second classes and the encoded representation; and
interpolating information in the semantic representation to obtain a third set of operating parameters from the plurality of sets of operating parameters.

34. The medium of claim 25, further comprises one of:
providing the optimal set of operating parameters to the aesthetic skin treatment unit, for controlling automated operation of the aesthetic skin treatment unit; or
displaying the optimal set of the operating parameter to a display unit associated with the aesthetic skin treatment unit, for manually controlling the operation of the aesthetic skin treatment unit.

35. The medium of claim 25, wherein providing the optimal set of operating parameters to the aesthetic skin treatment unit comprises:
correcting the preset operating parameters for performing the aesthetic treatment by the aesthetic skin treatment unit, in accordance with the optimal set of operating parameters.

36. The medium of claim 25, wherein determining the optimal set of operating parameters comprises:
calculating mean value of the plurality of sets of operating parameters to output optimal set of operating parameters.
